(19) United States
(12) Patent Application Publication
(10) Pub. No.: US 2012/0239080 A1
(43) Pub. Date: Sep. 20, 2012

(54) Laparoscopic Dual Grasper

(76) Inventor: Peter Fan, Saddle River, NJ (US)

(21) Appl. No.: 13/051,992

(22) Filed: Mar. 18, 2011

(51) Int. Cl. A61B 17/28

(52) U.S. Cl. 606/205

(57) ABSTRACT

Two laparoscopic graspers are merged, having one external sheath, one handle, two pairs of jaws, two actuating rods, and two controls. Two instruments may now be used in one hand, through one port, facilitating intra-corporeal knot tying. This new “laparoscopic dual grasper” is designed to enable the passing of one end of the suture around the opposite strand to make the required “throw”. This same instrument with the additional feature of the external shaft split into two halves, with a sliding connection, permits one of the two graspers to extend further, enabling two adjacent points on the same suture to be brought together, producing a loop, which is an alternate method of making the “throw” in the tying of a knot.

### FIELD OF THE INVENTION

A laparoscopic surgical instrument for intra-corporeal knot tying, with unique methods of making a throw or making a loop in the suture.

### BACKGROUND OF THE INVENTION

Tying of knots is essential in any kind of surgery, especially in laparoscopic surgery. The current art of laparoscopic knot tying employs either the extra-corporeal method, involving tying the knot by hand outside the body and pushing the knot inside with a knot pusher, which is time consuming, or the intra-corporeal method, involving manipulation of the suture with the tips of two laparoscopic graspers, which is technically difficult, tedious, cumbersome, challenging, requiring advanced skills, and has a long learning curve. Laparoscopic clip appliers, staplers, pre-tied knots and the like are useful substitutes, but they are expensive and have limitations. Despite considerable prior art, surgeons are still wanting a hand instrument that renders laparoscopic intra-corporeal knot tying easier and faster.

A discussion on knot tying would be more meaningful if the different parts of the suture are named: Here, the needle end of the suture is called the “Head End”. The opposite end is the “Tail End”. After the needle has passed through the tissue and popped off, the half of the suture nearest the Head End is the “Head Strand”. The other half is the “Tail Strand”.

There are two basic methods of tying a knot, whether done openly or laparoscopically. One method is making a “throw” which involves passing one end of a suture around its tail end. This is so simple when performed with the fingers, that everybody can do it, yet the literature on this subject fails to emphasize the four basic steps in making such a throw, which are: (1) pass the head end of the suture over and above the tail strand, making a cross, (2) pass the head end back under the tail strand, (3) transfer the head end from under the tail strand to above the tail strand, and (4) pass the head end back again over the tail strand. Of course the process could be the mirror image of the steps described above, namely the first step could be passing the head end under the tail strand, instead of above. A throw or tie will be produced after the strands are tightened up. The critical step is the third step, which involves release and re-grab of the suture. Note, that conventionally there is only one regular grasper available to carry out these four steps, as the grasper in the other hand is needed to steady the tail strand. This is the main reason for the present invention.

The other method is making a “loop”. In intra-corporeal knot tying, typically two graspers are used, one in each hand, and the commonest manner is using one grasper to wrap one end of the thread around the shaft of the second grasper, forming a loop, and the second grasper then pulls the other end of the thread through the loop. There have been many patents in the prior art that facilitate the making of a loop, but they are not simple or practicable. The invention here is named the “laparoscopic dual grasper”. It has two objects, one is passing the suture to make the throw, which becomes easier and faster when a second grasper is available simultaneously in the same port and held by the same hand. The second object is the easy fabrication of a loop, which is possible only with the sliding version of this instrument. It is obvious that making such a loop will also be easier and faster when a second grasper is available simultaneously in the same port. The present invention is a kind of “double grasper”. It is however totally different in design, character, purpose and use compared with other previous similar inventions.

Referring to the prior art, the Christoudias Double Grasper, has 3 jaws, with a common middle jaw, and functions well as a tissue approximator. The Ferzli Double Grasper, has a second pair of jaws positioned more proximally on the main shaft, and whose purpose is to anchor one end of a suture prior to twisting it around the shaft of the instrument in order to produce a loop. The Hasson Suture Tying Forceps, is similar to the Ferzli. The previous suture passers include—(a) the Arthrex Scorpion Suture Passer, an orthopedic instrument for passing sutures through hard tissue (b) the Arthrex Birdbeak Suture Passer, similar to the Scorpion, (c) the Goretex, the Aesculap, and other similar suture passers, which are used to pass sutures through a thickness of soft tissue such as the abdominal wall. There have been devices which pass the suture-needle side to side, for inserting sutures into tissues, which also help to tie a knot, e.g. (a) the Autosuture's Endo-stitch, (b) the Japanese Maniceps. Note these only pass the suture needle, not the suture thread per se. There have been devices that attempt to automatically tie a knot, e.g. Jerrigan's experimental rotating slotted disc in endo-cardiac surgery research. There have been many devices that help to create a loop, but each functions differently—(a) Kitano's grasper with the rotating sleeve, Japanese, (b) Donald Murphy's grasper with the extra horn, Australian, (c) Grice's sleeve catching instrument, (d) Bagnato & Wilson's device which simulates the radiological pig-tail catheter, with a preformed loop built into the tip of the catheter, which is deformable and purportedly a loop former, but it is difficult to manufacture and apply, and has not yet been reduced to practice, (e) Ferzli's double grasper, which anchors one end of the suture, as described above. The present invention however makes a loop in a unique manner, as described later. There have been devices using a pre-formed knot, (a) Ethicon's Endo-Loop, (b) the Duraknot, (c) LSI's device, (d) Pare's pre-tied knot, all of which do not help to tie knots. Even though the present invention is a somewhat complex instrument, its use is strictly confined to knot tying, and it is not claimed as a “multifunctional laparoscopic instrument”.

It should be noted that the vast majority of previous inventions related to intra-corporeal laparoscopic knot tying fail to address the basic problem of “how to make a throw with the graspers”. Rather, they side step the problem, and offer various alternatives, such as making multiple loops, similar to fishing knots, using pre-tied knots, knot pushers, suture clips, cinchers, tissue fasteners, anchors, stapling devices, etc.

There are two versions of the laparoscopic dual grasper as presented here. The one piece model has a single common external sheath, and is only good for suture passing to make the throw. The sliding model has a shaft that is split into two halves, which are interlocked, and in addition to being able to pass the suture, it is also capable of producing a loop in the suture. However when used for loop production, the sliding model may require the assistance of the other hand.

U.S. PATENT DOCUMENTS

| | | | | |
|---|---|---|---|---|
| 1. | 3,834,395 | Sep. 10, 1974 | Manuel Santos | 128/326 |
| 2. | 5,201,759 | Apr. 13, 1993 | George Ferzli. | |
| | 606/139 | | | |
| 3. | 5,217,471 | Jun. 8, 1993 | Stephen Burkhart | |
| | 606/148 | | | |
| 4. | 5,234,443 | Aug. 10, 1993 | Phan & Stoller | |
| | 606/148 | | | |
| 5. | 5,281,236 | Jan. 25, 1994 | Bagnato et. al. | |
| | 606/139 | | | |
| 6. | 5,312,423 | May 17, 1994 | Rosenbluth & | 606/148 |
| | | | Brenneman | |
| 7. | 5,395,382 | Mar. 7, 1995 | DiGiovanni et al. | |
| | 606/148 | | | |
| 8. | 5,437,682 | Aug. 1, 1995 | Drew Grice | |
| | 606/148 | | | |
| 9. | 5,423,836 | Jun. 13, 1995 | Scott Brown | |
| | 606/148 | | | |
| 10. | 5,439,467 | Aug. 8, 1995 | Theodore Benderev, | |
| | 606/139 | | et. al. | |
| 11. | 5,480,406 | Jan. 2, 1996 | Nolan et. al. | |
| | 606/139 | | | |
| 12. | 5,810,852 | Sep. 22, 1998 | Greenberg et. al. | |
| | 606/148 | | | |
| 13. | 5,814,054 | Sep. 29, 1998 | Kortenbach et. al. | |
| | 606/139 | | | |
| 14. | 5,846,254 | Dec. 8, 1998 | Schulze et.al. | |
| | 606/228 | | | |
| 15. | 6,051,006 | Apr. 18, 2000 | Shluzas & Sikora | |
| | 606/148 | | | |
| 16. | 6,086,601 | Jul. 1, 2000 | InBae Yoon | |
| | 606/148 | | | |
| 17. | 6,221,084 | Apr. 24, 2001 | R. Fleenor, Pare Surgical | |
| | 606/148 | | | |
| 18. | 6,432,118 | Aug. 13, 2002 | Mollenhauer & Kucklick | |
| | 606/148 | | | |
| 19. | 6,716,224 | Apr. 26, 2004 | Singhatat | |
| | 606/148 | | | |
| 20. | 2009/0228025 | Sep. 10, 2009 | Steven Benson | |
| | 606/144 | | | |
| 21. | 2010/0016883 | Jan. 21, 2010 | George Christoudias | |
| | 606/205 | | | |

### OTHER PUBLICATIONS

1. Endo-stitch—Autosuture—Manufacturer's item #173016.
2. Maniceps—Japanese suturing device, similar to Endo-stitch.
3. A Laparoscopic Device for Minimally Invasive Cardiac Surgery. (rotating slotted disc). Shaphan Jernigan, et. al. —European J. of Cardio-thoracic Surgery, Vol. 37, issue 3, p 626-630. March 2010.
4. Knot Tying Intra-corporeally, with newly designed Forceps. (sliding sleeve).
   Kitano et. al.—J. of Minimal Invasive Therapy & Allied Tech, 1996. 5: 27-28.
5. Endoscopic Knot Tying Made Easier—(one jaw with extra bump). Donald Murphy—ANZ J. Surg. 1995. 65, 507-509.
6. The Excalibur Suturing Needle Holder—(jaw with prominent heel, helps looping)
   Uchida et. al. Surgical Endoscopy—vol. 3, 531-532
7. Alijizawi laparoscopic auto-knot device—(two dissolving balls).
8. A New Reusable Instrument designed for simple and secure knot tying in laparoscopic surgery. S. S. Miller 1996 Surg. Endos 10: 940-941(pointed canula).
9. The Nobel Automatic Laparoscopic Knotting and Suturing Device. Mishra et. al. World Laparoscopy Hospital, India. (a knot pusher)
10. Automated Knot Tying for Fixation in Minimally Invasive Robot Assisted Cardiac Surgery. Kuniholm & Buckner—J. Biomed Eng. November 2005, Vol. 127, 1001-8.
11. JSLS. 2005 January-March; 9(1):105-12. M I Frecker
   Laparoscopic multifunctional instruments: design and testing . . . .
12. Endosc Surg Allied Technol. 1994 December; 2(6):318-9. G. Berci Multifunctional laparoscopic Instruments.
13. http://www.mri.psu.edu/articles/06s/MaryFrecker/index.asp
14. http://www.ligasure.com/ligasure/pages.aspx?page=Products/Laparoscopi
15. http://www.freepatentsonline.com/y2010/0063437.
16. http//www.ncbi.nlm.nih.gov/pubmed/15791983
Multifunctional Laparoscopic Instruments.
SUGGESTED U.S. CLASSIFICATION: 606/139, 144, 145, 148.
SUGGESTED INTERNATIONAL CLASSIFICATION: A61B 17/00, 04, 28.
FIELD OF SEARCH: 606/139, 144, 145, 147, 148, 150, 151, 127, 128, 606/167, 168, 170, 174, 182, 185, 205, 207, 210, 211.

### SUMMARY OF THE INVENTION

The laparoscopic dual grasper is derived from modification of the conventional single grasper, and is intended to make intra-corporeal knot tying easier and faster. It provides two graspers that can be used simultaneously in the same hand, and through the same port. This advantage means the surgeon may now use three instruments with two hands, and two ports. The purpose of the extra third grasper is to facilitate intra-corporeal suture manipulation. One object of the invention is to “pass” the head end of a suture from one side of the tail strand to the opposite side, to make the throw, using the same instrument and the same hand. Another object is to produce the necessary “loop” in the tail strand, which is another method of making a throw. The production of a loop however can only be made with the sliding version of this instrument, but the loop maneuver may require the assistance of the other hand. The instrument is designed with a pistol-like grip so the index finger would be free to control the second grasper. The second grasper unit may be entirely mounted and dismounted, so it may be made of disposable materials.

### BRIEF DESCRIPTION OF THE DRAWINGS

FIG. 1 is a perspective view of the device, showing the jaws, shaft, handle, trigger and cylinder.

FIG. 2 is a perspective view of the device, showing the internal assembly.

FIG. 3 is an exploded view of the upper (mother) grasper, showing the parts.

FIG. 4 is an exploded view of the lower (daughter) grasper, showing the parts.

FIG. 5 is left side view of the device, showing internal assembly.

FIG. 6 is left side view of the device, with cross section indicators.

FIG. 7A is cross section across the 4 jaws.

FIG. 7B is cross section of the assembled mother and daughter shafts.

FIG. 7C is cross section where the lower shaft joins the top of the cylinder.

FIG. 7D is cross section at the point where the peg passes through the cylinder.

FIG. 7E is cross section of the shaft if without the sliding mechanism.

FIG. 8A is top view and bottom view of the cylinder.

FIG. 8B is cross section of the cylinder.

FIG. 8C is side view of the cylinder.

FIG. 9 is prospective view of device, with the lower grasper extended forward.

FIG. 10 is left side view of the device, with the lower grasper extended forward.

FIG. 11 is left side view of the device with lower grasper retracted backwards.

FIG. 12 is prospective view of the upper and the lower graspers, separated.

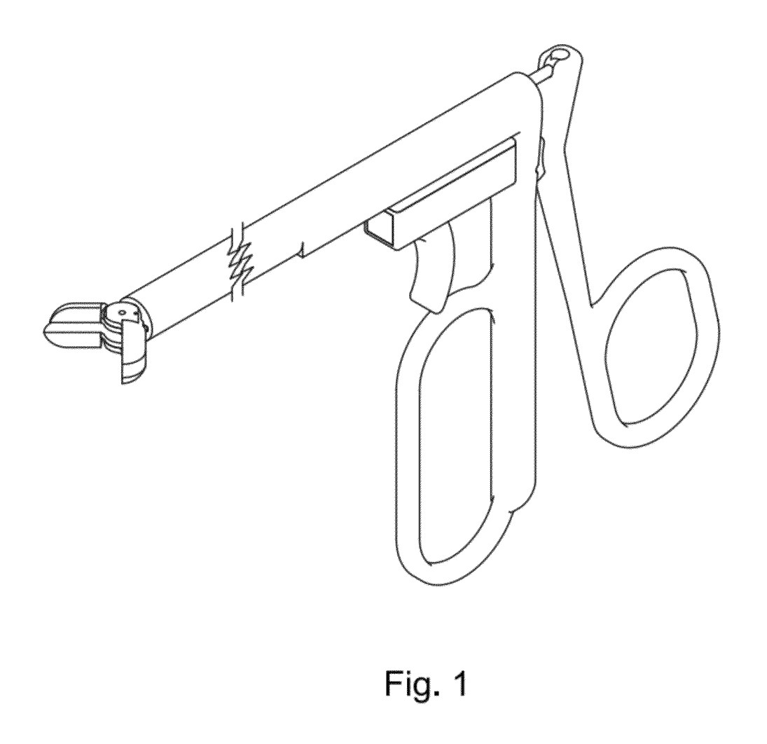

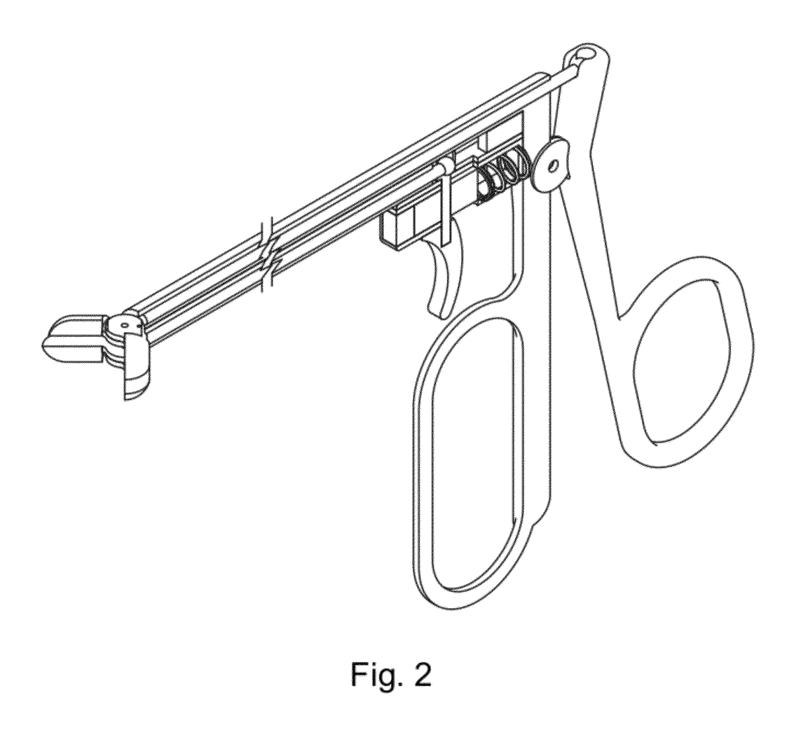

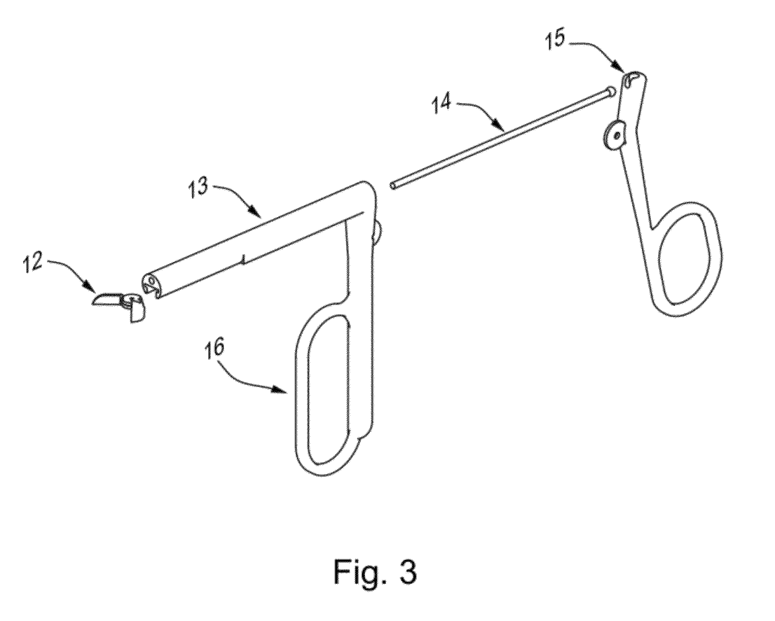

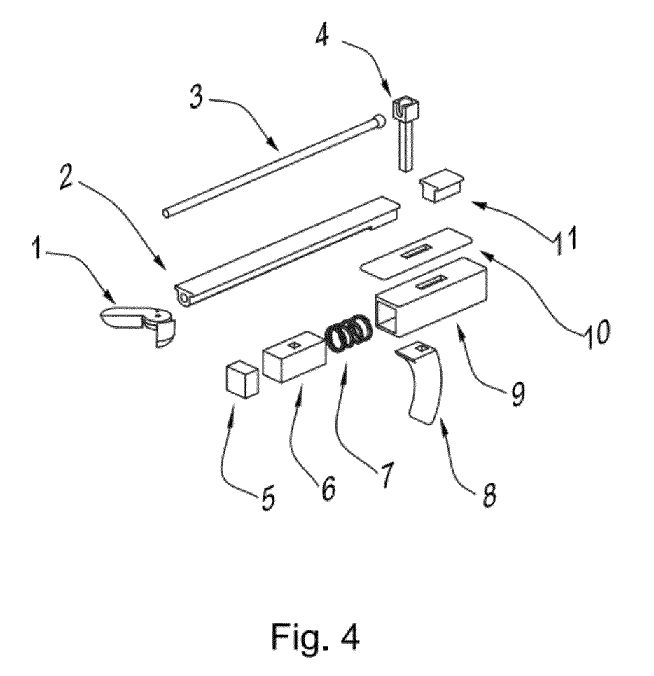

### NUMERAL REFERENCE TO PARTS IN FIGS. 4 & 5

| | | | |
|---|---|---|---|
| 1. | Jaws of lower grasper. | 2. | Shaft of lower grasper. |
| 3. | Rod of lower grasper. | 4. | Peg. |
| 5. | End plug of cylinder. | 6. | Piston. |
| 7. | Compression spring. | 8. | Trigger. |
| 9. | Cylinder. | 10. | Common Plate. |
| 11. | Posterior segment of lower shaft. | 12. | Jaws of upper grasper. |
| 13. | Shaft of upper grasper. | 14. | Rod of upper grasper. |
| 15. | Moving leg of upper grasper. | 16. | Stationary leg of upper |
| | | | grasper. |

### DETAILED DESCRIPTION OF THE PREFERRED EMBODIMENT

A laparoscopic surgical instrument, consisting of two graspers with one common external sheath, with two pairs of jaws, two actuating rods, and two actuating mechanisms. This arrangement permits the two graspers to be used simultaneously through the same port, with one hand. The two grasper systems are independent of each other, but their rods share the same external sheath.

An alternative construction involves splitting the entire shaft into two halves, then mating them to each other in a particular manner, and preserving a round exterior. One half shaft is slightly larger and hugs the smaller one, so the two are known as “mother” and “daughter” graspers.

The two sets of jaws are disposed one on top of the other, with their grasping surfaces in the vertical plane, so they open and close in the horizontal plane, whereas the jaws of the conventional grasper open and close in the vertical plane. The lower pair of jaws is slightly longer than the upper pair and protrudes out a little, to facilitate catching the tail strand. There is a small gap between the upper and lower pairs of jaws, which allows the tail strand to be temporarily trapped. This arrangement allows the head end of the suture to be passed from one side of the tail strand to the other side, which is essential in making the “throw”.

The actuating mechanism for the upper (mother) pair of jaws is similar to that in the conventional laparoscopic grasper. But the actuating mechanism for the lower (daughter) grasper requires special design to overcome certain constraints, one being the required close proximity between the upper and lower rods, another being the inability to make use of extension of the index finger, which extension is weak, thereby requiring the use of a trigger with a spring return. This trigger is located under the proximal part of the daughter shaft, for the convenience of the index finger, and is linked to the lower rod through the peg, so when the trigger is pulled by the index finger, the lower rod will be pulled back which in turn will close the lower jaws.

The entire daughter grasper may be dismounted, in which case the mother grasper may function as a regular single instrument, except its own shaft will no longer be round. The daughter grasper unit additionally may slide forwards upon the mother grasper, extending its reach, enabling the production of a loop in the suture, which is another technique in knot tying. Note the retraction of the daughter grasper goes more proximal than the original starting point, resulting in crossing over of the legs of the loop, which is essential in completing formation of the loop.

At rest both pairs of jaws of are designed to be in the open position. For the lower jaws the reason is that extension of the index finger is too weak. For the upper jaws the reason is they need to be in the open position to make room for the head end of the suture protruding up from the lower jaws. It is important that when grasping the head end of the suture with the lower jaws, one does not leave more than half inch protruding above the lower jaws. If the protruding portion is excessively long, it may prevent seating of the tail strand in the gap between the upper and lower jaws. Pulling the trigger will close the lower jaws, and flexing the thumb will close the upper jaws. Simultaneous action of the two, as indicated, will close all the jaws, presenting a blunt tip, and this position is to be maintained during entry into and exit from the trocar. The upper jaws can also be opened by extending the thumb or by pushing forward the top end of the ‘moving leg’ with the thumb. The upper jaws can be closed by flexing the thumb inside the thumb loop, or by squeezing forward the ‘moving leg’ using the thenar eminence of the palm. The end effectors on the dual grasper may be identical or different, or compound, such as grasping jaws combined with scissors, and may be interchangeable.

Recommended Steps in Suture Passing
1. Steady the tail strand with a regular grasper in the left hand.
2. Hold the dual grasper in the right hand, and hold all the jaws closed before entry.
3. After entry, release all the jaws which should be opened by the springs.
4. Point the dual grasper to the left, above and over the tail strand.
5. Use lower jaws of the dual grasper, to grab the head end of the suture, in such a manner that no more than half inch of it protrudes above the lower jaws.
6. Pull the dual grasper back over and above the tail strand.
7. Next, use the lower jaws of the dual grasper to lift up the tail strand, and trap it in the gap between the lower and upper jaws, until it is well seated. During this process, the lower jaws are closed, holding the head end, whilst the upper jaws remain open.
8. Next, close the upper jaws to take over the head end from the lower jaws, and pull away from the tail strand. As the index finger releases the trigger, the lower jaws will automatically open, due to the springs. This completes the throw.

Recommended Steps in Loop Forming
1. For a right handed person, hold the dual grasper in the left hand.
2. First grab anywhere about the mid portion of the tail strand with the upper jaws.
3. Next, slide out the lower grasper for about 2 inches.
4. With the lower grasper grab this same strand about 2 inches out.
5. Next, pull back the lower grasper, back beyond the starting point, so the two points on the suture cross over.
6. A loop will be formed. Make sure it is a ‘closed loop’. See explanation below.
7. Push the regular grasper in the right hand through this loop, grab the head end of the suture, and pull it back through the loop, and tighten up. A throw will be formed.
8. Release the jaws on the dual grasper. Then close all jaws and exit.

As explained above, the “loop” maneuver involves first using the mother grasper to grab anywhere along the middle of the “tail strand”, then push out the said daughter grasper, and use it to grab the same about 2 inches away. Upon retraction of the daughter grasper, as the two points on the strand are brought together and crossed over, a loop will be formed. However one must be careful to produce what is known as a ‘closed loop’, otherwise a useless ‘open loop’ might result. An ‘open loop’ is a progressive loop like a coiled spring. A ‘closed loop’ is where the returning portion of the suture reverses course and must end up on the ‘inside’ of the tie, i.e. next to the tissues to be enclosed within the tie.

With either the passing technique or the looping technique, to have the second throw end up as a square knot, the same maneuvers are repeated, except the naming of the strands and their treatment are reversed.

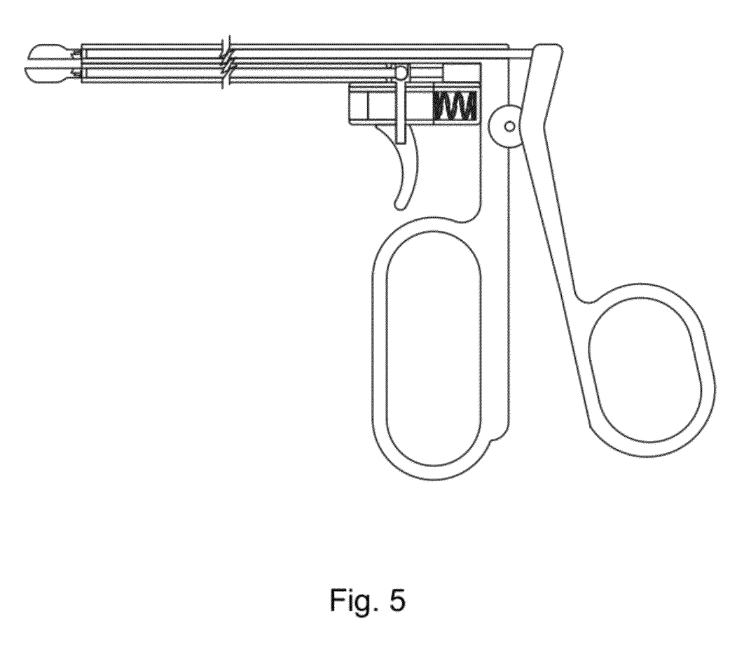

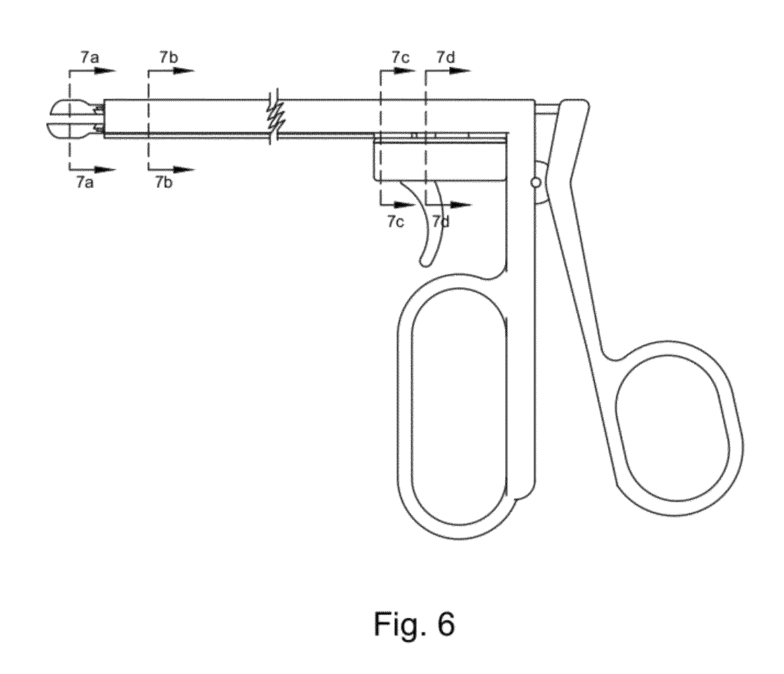

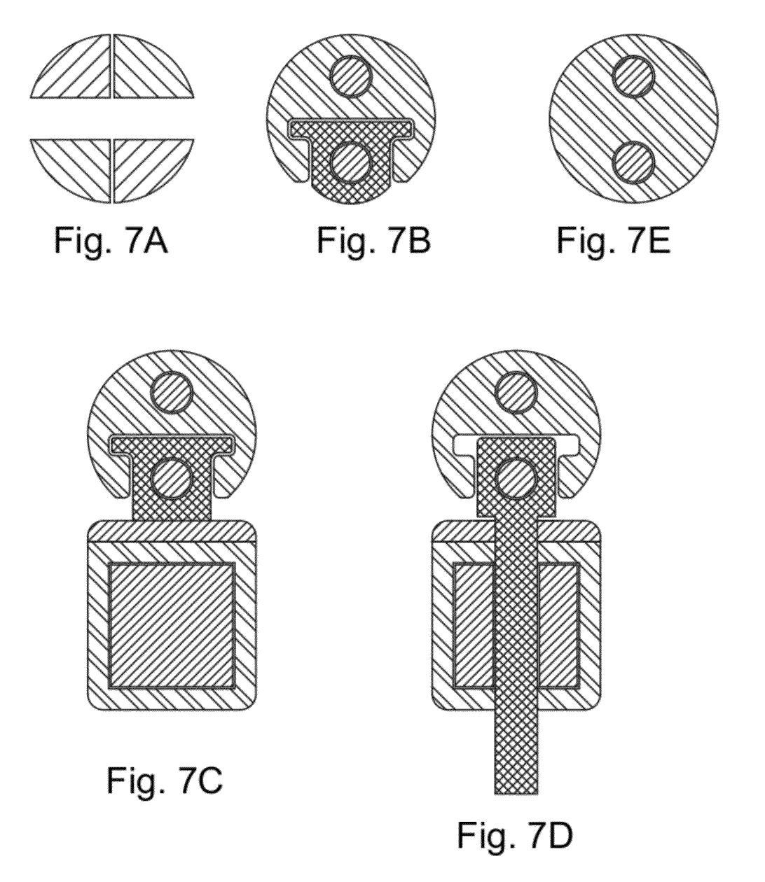

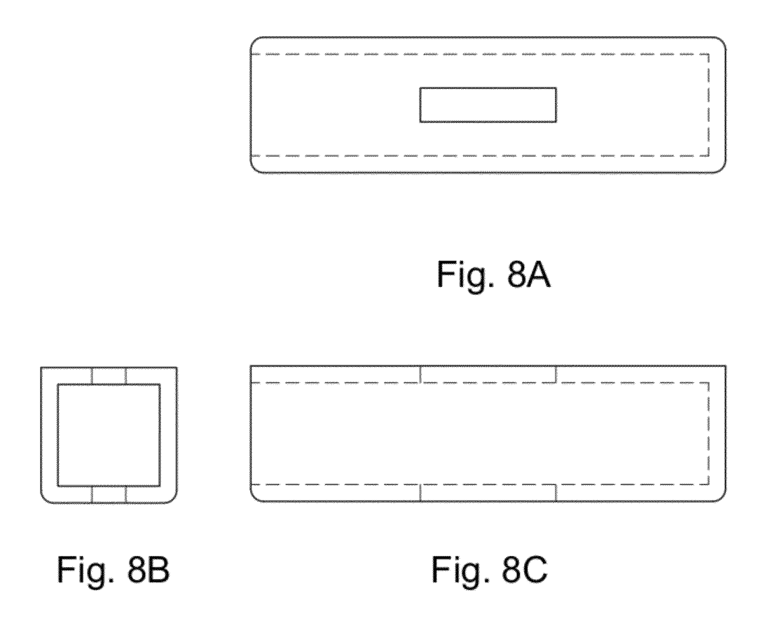

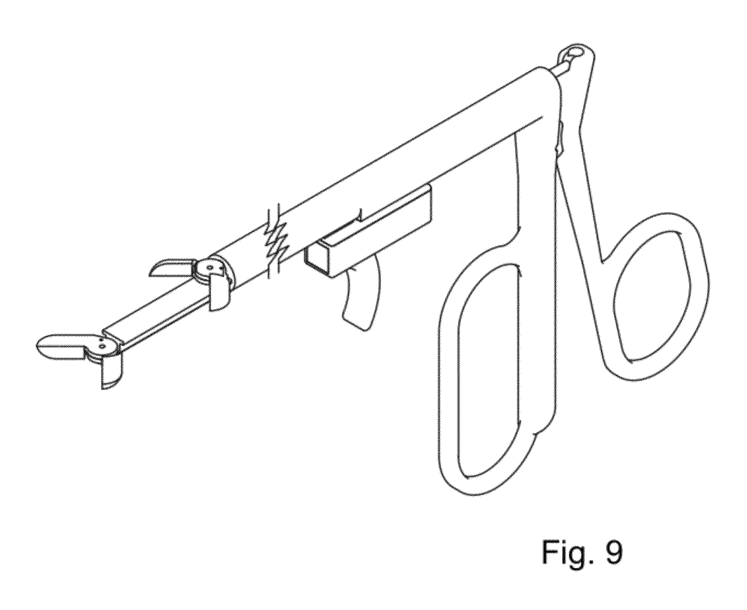

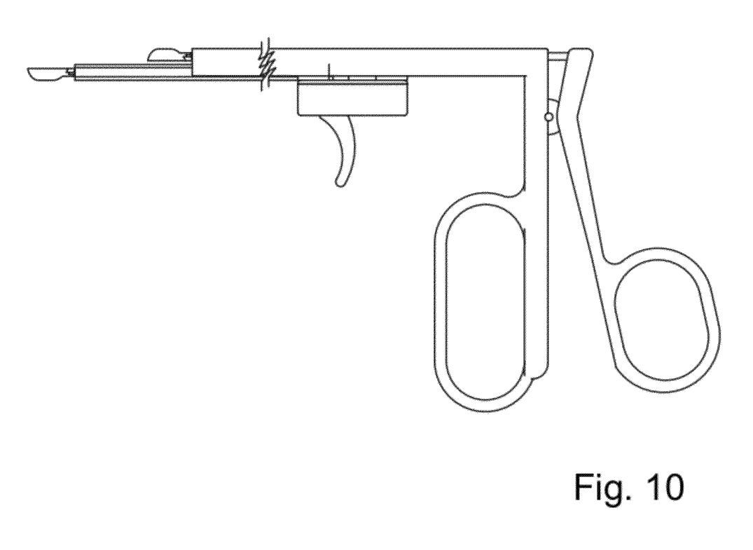

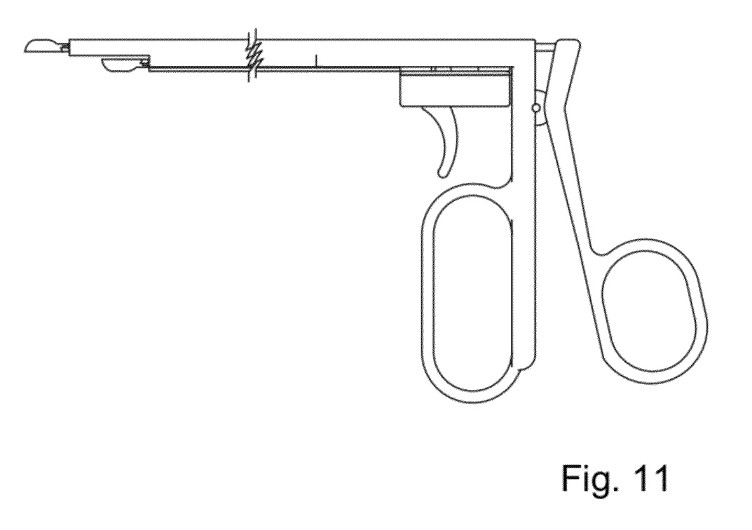

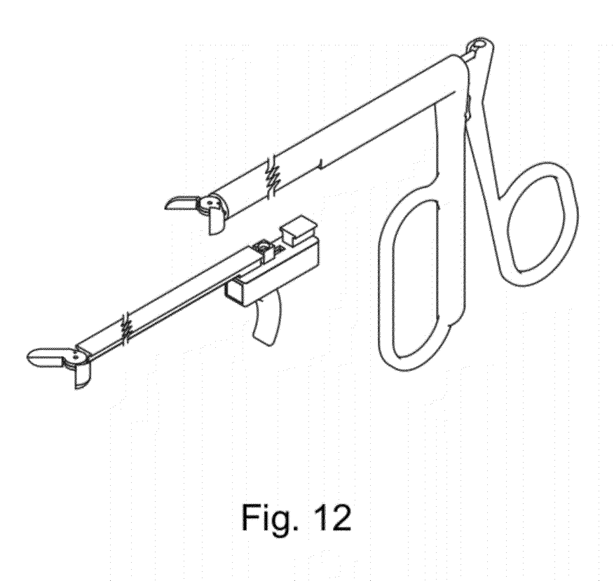

Referring to FIG. 5, an exploded view showing parts of the lower (daughter) grasper. The “lower jaws” #1, are the same as the upper jaws, except they are 1 mm longer. This allows the tail end of the thread to be scooped up, and then seated in the gap between the upper and lower pairs of jaws. The reason for the gap being 2 mm is that the head end of the suture can be easily passed to the receiving grasper. The grasping faces of the jaws are intentionally flat, which is best for grabbing the thread, whereas those with teeth may allow the thread to slip through. The “daughter shaft”, #2, is 35 cm. long, has a cross section resembling an inverted tomb-stone, and has ears on each upper lateral corner. Its external surface is curved, and it interlocks with the “mother shaft”, upon which it can slide forwards and back. The underside of its rear end is flat, to fuse with the front of the upper surface of the common plate. Behind its rear end there is a missing segment in order to provide space for the head of the peg, whose cross section simulates that of the daughter shaft minus the ears. The “free segment of daughter shaft”, #4, is needed to fuse with and to suspend the rear portion of the common plate, and hence also of the rear of the cylinder on the track above. It does not need a lumen. It does have ears. Its bottom is flat. The “end plug”, #5, is used to close off the compression spring and piston. The “piston”, #6, is a solid rectangular structure that sits inside the rectangular cylinder. The “piston” is 14 mm. L×6 mm. H×6 mm. W. The square peg passes through the cylinder and through the piston, but cannot fall out because the “head” of the peg is larger than the stem of the peg. A rectangular piston is stronger than a cylindrical one. The “compression spring”, #7, sits behind the rear of the piston. In the resting state, the compression spring holds the piston and the trigger in the forward position, which keeps the lower jaws open, without conscious effort from the operator. The “trigger”, #8, is a curved plate mounted to the bottom portion of the peg. The “cylinder”, #9, is an actuator for the lower (daughter) instrument. It is a rectangular box, contains the rectangular piston, the compression spring, the end plug, and the operating peg. The top and bottom surfaces of the cylinder show a slot, 2 mm wide×8 mm long, within which the peg travels. The external dimensions of the cylinder measure: 28 mm L×8 mm H×8 mm W. The upper surface of the cylinder is flat, and mates with the bottom surface of the common plate. The “common plate”, #10, is 2 mm thick. It has the same length and width as the upper surface of the cylinder, also has a 2 mm wide×10 mm long slot for travel of the peg. It has an important role in joining the rear end of the daughter shaft to the free segment behind the head of the peg. The first step in assembly is to insert the lower rod, #3, into the lower shaft, #2. Next the head of the peg is inserted up to house the ball at the posterior end of the lower rod. Next the common plate is inserted up the bottom end of the peg. Next, parts #2 and #11 are fused with the upper surface of the common plate. Next, the assembled cylinder is pushed up the bottom end of the peg. Next, the upper surface of the cylinder is fused with the bottom surface of the common plate. Finally, the trigger, #8, is pushed up the bottom end of the peg. The “operating peg”, #11, is basically square in cross section from top to bottom. The “head” at the upper end of the peg is larger in cross section than the lower portion passing through the piston, and measures 4 mm×4 mm×4 mm. The anterior surface of the ‘head’ is notched 2 mm wide, ×3 mm deep. Its upper end is bored, 3 mm diameter, centered, and 3 mm deep, communicating with the notch. It receives the “metal ball” at the rear end of the lower shaft, and forms a ball joint. The lower portion of the peg, is 2 mm×2 mm in cross section, and passes through the piston, ending in the center of the “trigger”. A détente mechanism provides some friction between the opposing surfaces of the mother and daughter shafts to enable the latter to remain at the position it was set. This consists of a small metal ball, a compression spring, and a threaded thumb screw, which are all inserted up a tunnel within the cap at the rear end of the cylinder, and up through the free segment of the daughter shaft.

Referring to FIG. 4, the “upper jaws”, #12, are 1 mm shorter than the lower jaws. The “mother shaft”, #13 (upper shaft), is 35 mm long, and embraces the daughter shaft (lower shaft). Its outer surface is mostly round. Its cross section is basically round, 8 mm. diameter, but with the bottom third missing, and shows the embracing arms at the sides. There is a lumen, 2 mm. diameter passing through its long axis for the passage of the “upper rod”. The underside of its rear end is flat and is fused with the upper end of the stationary leg, and the two are in one piece. The “stationary leg” #16, has a stem 75 mm long. Its lower 50 mm. is part of the large finger loop. Its cross section is 8 mm×8 mm, and this extends from the upper end all the way down to the lower end, and is intended to provide a firmer grip. The large finger loop, has an ID of 20 mm horizontal×50 mm vertical, and except for the vertical portion that is gripped by the fingers, its cross section is 3 mm×3 mm, all with rounded corners. The stem is at 90 degrees to the mother shaft, to resemble a pistol grip. The finger loop is positioned anterior to the stem. On the back side of the stem about 12 mm from the top, is a round partially protruding flange, 9 mm diam. and 6 mm thick. This flange contains a 3 mm diam. hole from side to side in its center (for a 3 mm diam. screw). The center of the hole is 15 mm below the upper surface of the main body. The center third of the vertical thickness of this flange is cut out, converting it into two side by side flanges, each 2 mm thick, with a gap of 2 mm between them, to receive the circular mating male flange of the moving leg. The “upper rod”, #14, is conventional, is 2 mm diameter, with a small metal ball at its rear end. The attachment at its front end to the jaws is conventional. The “moving leg”, #15, articulates with the stationary leg and is positioned in the same plane behind the stationary leg. Its stem is divided into two portions, an upper portion above and a lower portion below the center of the flange. Both portions are each bent backwards for 35 degrees, at the level of the flange. The upper part is 16 mm long, the lower part is 32 mm long, down to the top of the thumb loop. The cross section is 6 mm×6 mm at its upper end, narrowing to 4 mm×4 mm at its lower end which fuses with the loop. This loop has an ID of 20 mm horizontal, ×25 mm vertical, and a cross section of 3 mm×3 mm, with rounded edges, and is positioned posterior to the stem. On the anterior aspect of the upper part of the stem, 14 mm below the upper end, is the center of a semi-circular protruding flange, with 4 mm radius, 2 mm thick. On each lateral surface of the stem adjacent to the flange is a semi-circular cut out 2 mm deep, also 4 mm radius, which narrows the flange to a 2 mm thick male piece. There is 2 mm diam. hole in the center of the flange. The anterior surface of its upper end bears a notch 2 mm wide, ×3 mm deep. The upper surface of its upper end bears a round drill-out 3 mm diam.×3 mm deep, communicating with the notch, to form a ball joint. The inside distance between the two legs at a point half way down the smaller loop, is 25 mm when closed, and 40 mm when open. The upper end of the moving leg is rounded, which allows it to be pushed forwards by the thumb, which will have the same effect as opening the moving leg away from the stationary leg. The “torsion spring”, #17, is conventional, and is not shown. The spring holds the moving leg backwards which keeps the upper jaws open, but is removable. The “screw”, #18, is conventional and is not shown. Overall length of screw is 6 mm, with a 2 mm thick head, containing a sunken slot, with a shaft 3 mm diam. with a blunt tip. The screw holds the torsion spring in place and holds the moving leg to the stationary leg.

What is claimed:

1. A laparoscopic surgical instrument for intra-corporeal knot tying, the laparoscopic dual grasper, resulting from the merging of two laparoscopic graspers, comprising: two pairs of grasping jaws; two actuating rods; two actuating controls; one common external sheath; and one handle, wherein the said instrument may to be used through one port, and with one hand.

2. The instrument of claim 1, wherein the said two pairs of grasping jaws are adjacent, parallel to each other, side by side, separated from each other by a gap, with one pair of the jaws longer than the other, wherein a throw in the tie is enabled remotely.

3. The instrument of claim 1, wherein all the jaws of the said instrument open and close in the horizontal plane, with each jaw occupying a quadrant of a circle, so that when all the 4 jaws are in the closed position, a blunt tip is presented.

4. The instrument of claim 1, wherein the said two actuating rods are adjacent, parallel to each other, side by side, and are both contained within a common external sheath, permitting the use of one port.

5. The instrument of claim 1, wherein the actuator for one grasper is operated by the thumb and the other grasper is operated independently by the index finger, thereby permitting the said instrument to be used with one hand.

6. The instrument of claim 1, wherein the stem of the stationary leg of the said handle, is positioned at 90 degrees to the said common external sheath, and the cross section of the said stem is sufficiently large, so as to enable a firm grip by the palm, the middle and ring fingers.

7. The method of creating a throw in the suture, as provided by the instrument of claim 1, wherein the head end of a suture is transferred from a grasping mechanism on one side of the tail strand of the suture, to an adjacent and parallel second grasping mechanism on the opposite side of the tail strand, a necessary step in the throw.

8. The instrument of claim 1, in an alternative construction, without the shared common external sheath, comprises: the said two pairs of grasping jaws; the said two actuating rods; the said two actuating controls; the said one handle; and two separate shafts further comprising: one of the said actuating rods within each shaft; with the said two shafts interlocking with each other; with the said combination preserving a round exterior; permitting one shaft to slide upon the other; extending its reach; with either of the said shafts and all its connected components capable of being dismounted, discarded and replaced.

9. The instrument according to claim 7, wherein the interlocking joint between the said two shafts is T-shaped, with the cross section of one shaft being in the shape of an inverted head-stone, and that of the other shaft being complimentary, with embracing side arms.

10. The instrument according to claim 7, wherein the actuator mechanism of the said sliding member, is totally attached to the sliding member, and is able to move with it.

11. The method of creating a loop in the suture by approximating two adjacent points of the suture, as performed by the instrument according to claim 7, and similarly the approximation or distraction or the simultaneous pulling of two adjacent points of tissue.

12. It will be obvious to the experienced eye, or to those skilled in the art, that many variations from this basic design are possible and these may include the following: In the one piece non-sliding version, variations are possible in the dimensions of the components of the device; in the design and shape of the jaws; the jaws could be angled, or able to flex; in the combination of the jaws and other possible end effectors, such as scissors; in the design of the actuating mechanism which could be replaced by a simple flat spring; the said gap between the said two pairs of jaws may be smaller or greater than the prescribed 2 mm; the outside diameter of the common external shaft may vary from 5 mm to 10 mm; the axial alignment of the two rods instead of being at 12 & 6 o'clock, may be at 9 & 3 o'clock or any other such combination; and in the sliding version of the said instrument, variations are possible in the shape and design of the interlocking mechanism between the two said shafts.
